(19) United States
(12) Patent Application Publication
(10) Pub. No.: US 2024/0139100 A1
(43) Pub. Date: May 2, 2024

(54) TRANSMUCOSAL DELIVERY OF TOCOTRIENOLS

(71) Applicant: INVICTUS BIOTECHNOLOGY PTY LTD, Melbourne, Victoria (AU)

(72) Inventors: Glenn TONG, Melbourne, Victoria (AU); David KINGSTON, Melbourne, Victoria (AU)

(21) Appl. No.: 18/255,796

(22) PCT Filed: Dec. 3, 2021
(86) PCT No.: PCT/AU2021/051449

§ 371 Date: Jun. 2, 2023

(30) Foreign Application Priority Data
Dec. 4, 2020 (AU) 2020904488

(51) Int. Cl. A61K 9/00; A61K 31/355; A61K 47/02; A61K 47/10; A61K 47/26; A61K 47/36

(52) U.S. Cl. CPC A61K 9/006; A61K 31/355; A61K 47/02; A61K 47/10; A61K 47/26; A61K 47/36

(57) ABSTRACT

The present disclosure relates to a formulation comprising tocotrienols and derivatives thereof, for transmucosal (such as buccal, sublingual and mucosal) administration.

### CROSS-REFERENCE TO RELATED APPLICATIONS

This application is the U.S. national phase of International Application No. PCT/AU2021/051449 filed Dec. 3, 2021, which designated the U.S. and claims priority to AU2020904488 filed 4 Dec. 2020, the entire contents of each of which are hereby incorporated by reference.

### TECHNICAL FIELD

The present disclosure relates to a formulation comprising tocotrienols and derivatives thereof, for transmucosal (such as buccal, sublingual and mucosal) administration.

### BACKGROUND

An essential nutrient for the body, vitamin E is made up of four tocopherols (alpha, beta, gamma, delta) and four tocotrienols (alpha, beta, gamma, delta), with the difference between tocotrienols and tocopherols lying in the unsaturated side chain having three double bonds in its farnesyl isoprenoid tail for tocotrienols whereas these double bonds are single bonds in the tocopherols (FIG. 1).

Tocotrienols occur in selected vegetable oils such as palm and rice bran, certain types of fruits such as annatto and saw palmetto, nuts such as macadamia and plant products such as rubber tree latex. The tocotrienol component of the total vitamin E is generally lower than the tocopherol component.

Chemically, each of the tocotrienol and tocopherol isomers have an antioxidant activity due to their ability to donate a hydrogen atom (a proton plus electron) from the hydroxyl group on the chromanol ring to a free radical in the body. This process inactivates (“quenches”) the free radical by effectively donating a single unpaired electron (which comes with the hydrogen atom) to the radical.

Vitamin E has long been known for its antioxidative properties against lipid peroxidation in biological membranes and alpha-tocopherol has previously been considered to be the most active form. However, in vivo, tocotrienols are more powerful antioxidants, and lipid oxygen radical absorbance capacity (ORAC) values are highest for delta-tocotrienol. Recent data would suggest that tocotrienols are better antioxidants than tocopherols at preventing cardiovascular diseases and cancer, and in the treatment of diabetes. Current formulations of vitamin E supplements which are commercially available are composed mainly of alpha-tocopherol.

Tocotrienols have many uses beyond their lipid-soluble antioxidant property. They specifically inhibit biosynthesis of cholesterol by the liver through enhanced degradation of the enzyme HMG-CoA reductase (Song et al “Insig dependent ubiquitination and degradation of 3-hydroxy-3-methylglutaryl coenzyme a reductase by delta- and gamma-tocotrienols” The Journal of Biological Chemistry 281 (35):25054-61). Tocotrienols have been shown to inhibit inflammatory pathways mediated by NF-κB (Nesaretnam et al “Tocotrienols: inflammation and cancer” Ann N YAcad Sci. 2011 July; 1229:18-22). They have also been identified as agonists to peroxisome proliferator-activated receptor (PPAR), in particular PPAR-gamma, which is an insulin-sensitiser in addition to increasing adipogenesis (Fang et al “Vitamin E tocotrienols improve insulin sensitivity through activating peroxisome proliferator-activated receptors” Mol Nutr Food Res 2010 March; 54(3): 345-52). Indeed, tocotrienols influence many more biochemical pathways than tocopherols, and are being developed as treatments for inflammation, ischemia-associated diseases such as stroke and myocardial infarct, dyslipidaemia and even cancer (Khosia et al “Postprandial levels of the natural vitamin E tocotrienol in human circulation” Antioxidants & Redox Signalling 8(5-6): 1059-68). Tocotrienols have been shown to or have the potential to: (i) have strong antioxidant properties (Serbinova et al “Free radical recycling and intramembrane mobility in the antioxidant properties of alpha-tocopherol and alpha-tocotrienol” Free Radical Biology & Medicine 10(5):263-75); (ii) reverse hypertension and cardiac fibrosis (Black et al “Palm tocotrienols protect ApoE+/− mice from diet induced atheroma formation” J Nutrition 2000; 130(10):2420-6); (iii) improve control of blood glucose and insulin response (Kuhad et al (2009) “Suppression of NF-κB signalling pathway by tocotrienol can prevent diabetes associated cognitive defects” Pharmacology Biochemistry, and Behaviour 92(2):251-9); (iv) specifically inhibit biosynthesis of cholesterol by the liver, i.e., they can lower cholesterol levels and ameliorate dyslipidaemia (Song et al “Insig dependent ubiquitination and degradation of 3-hydroxy-3-methylglutaryl coenzyme a reductase by delta- and gamma-tocotrienols” The Journal of Biological Chemistry 281 (35):25054-61); (v) inhibit inflammatory pathways mediated by cyclooxygenase-2 and 12-lipoxygenase, i.e., they can be used as treatments for inflammation (Khanna et al “Molecular basis of vitamin E action: tocotrienol modulates 12-lipoxygenase, a key mediator of glutamate induced neurodegeneration” J Biol Chem 2003; 278:43508-43515); (vi) potentially be useful as treatments for stroke, myocardial infarct, and even cancer (Hussein et al “d-Delta-tocotrienol-mediated suppression of the proliferation of human PANC-1, MIA PaCa-2, and BxPC-3 pancreatic carcinoma cells” Pancreas 38(4):e124-36); (vii) improve exercise endurance and improve muscle glycogen levels (Lee et al “Effects of tocotrienol-rich fraction on exercise endurance capacity and oxidative stress in forced swimming rats” Eur J Appl Physiol 2009; 107(5):587-95); and (viii) act as radioactive countermeasures for persons exposed to radiation (Ghosh et al “Gamma-tocotrienol, a tocol antioxidant as a potent radioprotector” Int J Radiat Biol 85(7):598-606).

Dietary lipids and fat-soluble vitamins, such as vitamin E, to be absorbed from the gastrointestinal tract they must first be emulsified by bile and packaged into micelles for transport into the circulation. Bile excretion is dependent on the level and type of dietary fat consumed, and studies have shown that tocotrienol absorption is reduced in fasted versus full-fed individuals (Yap et al “Pharmacokinetics and bioavailability of alpha-.gamma- and delta-tocotrienols under different food status” J Pharm Pharmacol 2001 January; 53(1):67-71). Oral administration of isolated tocotrienols by gavage or gel capsules may therefore lack sufficient fat content to stimulate enough bile excretion into the small intestine that would be necessary to promote tocotrienol absorption. Following oral administration, tocotrienols are absorbed from the intestine and transported to the systemic circulation through the lymphatic pathway.

Although various tocotrienol-containing products are already commercially available, these products are typically capsules filled with a blend of various tocopherols and tocotrienol oils and sold as nutritional supplements for oral consumption. This type of formulation or delivery system displays poor solubility in the fluids of the intestine and high oral doses of tocotrienols inhibit its own absorption from the gut. Consequently, only relatively low levels of tocotrienol will reach the blood.

Attempts have been made to improve the bioavailability of tocotrienols. One strategy that is in current use is to use an emulsifying agent to enhance absorption from the gastrointestinal tract. A second strategy involves incorporating them into lipid nanoparticles or transferrin-bearing multilamellar vesicles, which appears to enhance the antitumor effect of tocotrienols by up to 70-fold (Fu et al, “Novel tocotrienol-entrapping vesicles can eradicate solid tumours after intravenous administration” J Control Release 2011 Aug. 25; 154(1):20-6). However, such formulations are limited in that they must be introduced intravenously (which is not practical or suitable for non-clinical applications and have limited market acceptance for all but the most serious and life-threatening therapeutic indications) and are dependent upon the use of tocopheryl based multilamellar vesicles which may themselves interfere with the activity of the tocotrienols present.

Given the potential clinical benefits of tocotrienols, and their low toxicity (Nakamura et al, “Oral Toxicity of a tocotrienol preparation in rats” Food Chem Toxicol 2001 August; 39(8): 799-805), there is a need for alternative formulations of tocotrienols, for example formulations that provide higher bioavailability than has been possible to date and/or formulations that provide one or more advantages over previously described formulations.

### SUMMARY

The present disclosure provides a formulation for oral transmucosal administration of at least one tocotrienol or derivative thereof, comprising:
a first composition comprising at least one tocotrienol or a derivative thereof, starch or a derivative of thereof, and silicon dioxide; and
a second composition comprising one or more excipients.
wherein the first composition and the second composition are combined to form the formulation.

In some embodiments, the one or more excipients comprise a mucoadhesive polymer selected from the group consisting of lectin, an acrylate, a hyaluronic acid, an alginate, a gellan gum, a poloxamer, a polyethylene glycol, a pectin, a starch, a sulfated polysaccharide, a gelatin, a chitosan, a Carrageenan, and a cellulose derivative and combinations thereof. In some embodiments, the one or more excipient comprises polyethylene glycol. In some embodiments, the one or more excipients comprises polyethylene glycol 8000.

In some embodiments, wherein the one or more excipients comprise a binder, bulking agent, diluent, pore former, lubricant, surfactant, disintegrant, buffer, sweetener or flavour or combination thereof.

In some embodiments, wherein the formulation is a solid dosage form. In some embodiments, the solid dosage form is a tablet, lozenge, wafer, a pill, a capsule, a membrane, a strip, a patch, a film, or a powder. In some embodiments, the solid dosage form is a powder or a tablet. In some embodiments, the solid dosage form is a powder.

In some embodiments, the at least one tocotrienol is selected from the group consisting of alpha-tocotrienol, beta-tocotrienol, gamma-tocotrienol, delta-tocotrienol and combinations thereof. In some embodiments, the at least one tocotrienol comprises delta-tocotrienol.

In some embodiments, the starch or a derivative thereof present in the first composition is tapioca dextrin. In some embodiments, the starch or a derivative thereof present in the first composition is modified food starch.

In some embodiments, wherein the first composition comprises between 0.1%-2.5% w/w silicon dioxide.

In some embodiments, the first composition comprises between 40%-60% w/w starch or derivative thereof.

In some embodiments, the first composition comprises between 2 to 50% w/w total tocotrienol, preferably 35% w/w total tocotrienol. In some embodiments, the formulation comprises between 2 to 10% w/w total tocotrienol.

In some embodiments, the formulation comprises mannitol.

In some embodiments, the formulation comprises one or more tocotrienol, silicon dioxide, dextrin, polyethylene glycol 8000, partially pregelatinized corn starch, mannitol, a flavour and a sweetener.

In some embodiments, the formulation consists of one or more tocotrienol, silicon dioxide, dextrin, polyethylene glycol 8000, partially pregelatinized corn starch, mannitol, a flavour and a sweetener.

In some embodiments, the transmucosal administration is sublingual and/or buccal.

The present disclosure also provides a process for preparing a formulation for oral transmucosal administration of at least one tocotrienol or derivative thereof, comprising:
(a) combining at least one tocotrienol or a derivative thereof, silicon dioxide and starch or a derivative thereof to form a first composition; and
(b) combining the first composition with one or more excipients.

In some embodiments, the one or more tocotrienol is an oil.

In some embodiments, combining the first composition with one or more excipients comprises mixing or agitation to form a homogeneous and free flowing powder. In some embodiments, combining the first composition with one or more excipients comprises mixing at a speed of 25 to 35 rpm for 20 to 30 minutes.

In some embodiments, the process further comprises:
(c) forming a solid dosage form selected from a lozenge, a pill, a tablet, a capsule, a membrane, a strip, a patch, a film, or a powder.

The present disclosure also provides a method of treating or preventing a disease or condition amenable to treatment with a tocotrienol comprising transmucosal administration of the formulation described herein. In some embodiments, the disease or condition is selected from the group consisting of post exercise muscle soreness, delayed onset muscle soreness, muscle recovery after exercise, maintenance of peak muscle power, fibrosis, hypertension, inflammation, stroke, cancer, elevated cholesterol and/or triglycerides; baldness, hypertrophy and a condition resulting from radiation exposure.

The present disclosure also provides a method of stabilizing and/or controlling blood glucose levels in a subject comprising transmucosal administration of the formulation described herein.

The present disclosure also provides a method of improving exercise endurance in a subject comprising transmucosal administration of the formulation described herein.

The present disclosure also provides a method of improving exercise capacity in a subject comprising transmucosal administration of the formulation described herein.

The present disclosure also provides a method of improving muscle recovery after exercise in a subject comprising transmucosal administration of the formulation described herein.

The present disclosure also provides a method of improving the maintenance of peak muscle power in a subject comprising transmucosal administration of the formulation described herein.

In some embodiments, the transmucosal administration is sublingual administration.

The present disclosure also provides use of the formulation described herein for the manufacture of a medicament for the treatment of a disease or condition amenable to treatment with a tocotrienol, wherein the formulation is formulated for transmucosal administration. In some embodiments, the disease or condition is selected from the group consisting of post exercise muscle soreness, delayed onset muscle soreness, muscle recovery after exercise, maintenance of peak muscle power, fibrosis, hypertension, inflammation, stroke, cancer, elevated cholesterol and/or triglycerides; baldness, hypertrophy and a condition resulting from radiation exposure.

The present disclosure also provides the formulation described herein for use in the treatment of a disease or condition amenable to treatment with a tocotrienol, wherein the formulation is formulated for transmucosal administration. In some embodiments, the disease or condition is selected from the group consisting of post exercise muscle soreness, delayed onset muscle soreness, muscle recovery after exercise, maintenance of peak muscle power, fibrosis, hypertension, inflammation, stroke, cancer, elevated cholesterol and/or triglycerides; baldness, hypertrophy and a condition resulting from radiation exposure.

### BRIEF DESCRIPTION OF DRAWINGS

FIG. 1: Shows the structure of the most common tocopherols and tocotrienols.

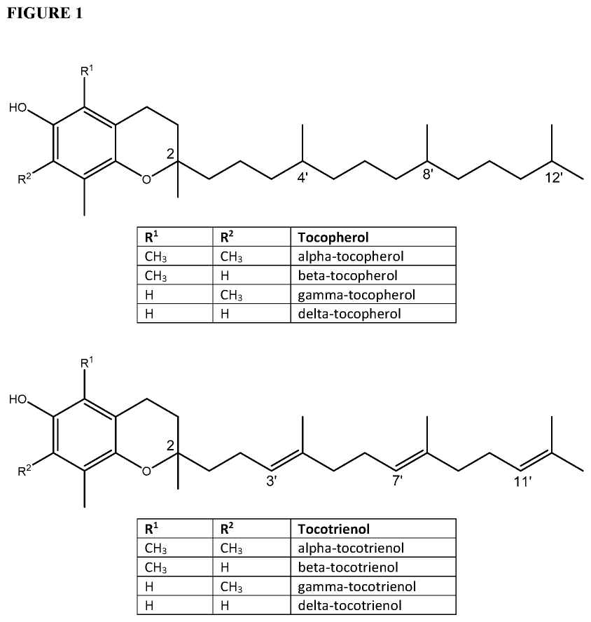

FIG. 2: Plasma tocotrienol concentration after sublingual administration of exemplified formulation to nine rats (three groups at three dosages of 1 mg/kg, 3 mg/kg and 6 mg/kg).

FIG. 3: Plasma tocotrienol concentration after sublingual administration of exemplified formulation to human subjects.

### DESCRIPTION OF EMBODIMENTS

### Terms

With regards to the definitions provided herein, unless stated otherwise, or implicit from context, the defined terms and phrases include the provided meanings. Unless explicitly stated otherwise, or apparent from context, the terms and phrases below do not exclude the meaning that the term or phrase has acquired by a person skilled in the relevant art. The definitions are provided to aid in describing particular embodiments, and are not intended to limit the claimed invention, because the scope of the invention is limited only by the claims. Furthermore, unless otherwise required by context, singular terms shall include pluralities and plural terms shall include the singular.

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

Throughout this disclosure, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the examples, steps, features, methods, compositions, coatings, processes, and coated substrates, referred to or indicated in this specification, individually or collectively, and any and all combinations or any two or more of said steps or features.

The term “and/or”, e.g., “X and/or Y” shall be understood to mean either “X and Y” or “X or Y” and shall be taken to provide explicit support for both meanings or for either meaning.

Unless otherwise indicated, the terms “first,” “second,” etc. are used herein merely as labels, and are not intended to impose ordinal, positional, or hierarchical requirements on the items to which these terms refer. Moreover, reference to a “second” item does not require or preclude the existence of lower-numbered item (e.g., a “first” item) and/or a higher-numbered item (e.g., a “third” item).

As used herein, the phrase “at least one of”, when used with a list of items, means different combinations of one or more of the listed items may be used and only one of the items in the list may be needed. The item may be a particular object, thing, or category. In other words, “at least one of” means any combination of items or number of items may be used from the list, but not all of the items in the list may be required. For example, “at least one of item A, item B, and item C” may mean item A; item A and item B; item B; item A, item B, and item C; or item B and item C. In some cases, “at least one of item A, item B, and item C” may mean, for example and without limitation, two of item A, one of item B, and ten of item C; four of item B and seven of item C; or some other suitable combination.

As used herein, the term “about”, unless stated to the contrary, typically refers to +/−10%, for example+/−5%, of the designated value.

It is to be appreciated that certain features that are, for clarity, described herein in the context of separate embodiments, may also be provided in combination in a single embodiment. Conversely, various features that are, for brevity, described in the context of a single embodiment, may also be provided separately or in any sub-combination.

Throughout the present specification, various aspects and components of the invention can be presented in a range format. The range format is included for convenience and should not be interpreted as an inflexible limitation on the scope of the invention. Accordingly, the description of a range should be considered to have specifically disclosed all the possible sub-ranges as well as individual numerical values within that range, unless specifically indicated. For example, description of a range such as from 1 to 5 should be considered to have specifically disclosed sub-ranges such as from 1 to 3, from 1 to 4, from 1 to 5, from 2 to 4, from 2 to 5, from 3 to 5 etc., as well as individual and partial numbers within the recited range, for example, 1, 2, 3, 4, 5, 5.5 and 6, unless where integers are required or implicit from context. This applies regardless of the breadth of the disclosed range. Where specific values are required, these will be indicated in the specification.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The reference to “substantially free” generally refers to the absence of that compound or component in the composition other than any trace amounts or impurities that may be present, for example this may be an amount by weight % in the total composition of less than about 1%, 0.1%, 0.01%, 0.001%, or 0.0001%. The formulations and compositions as described herein may also include, for example, impurities in an amount by weight % in the total composition of less than about 5%, 4%, 3%, 2%, 1%, 0.5%, 0.1%, 0.01%, 0.001%, or 0.0001%.

The term “formulation” or “or “dosage form” as used herein refers to a composition containing at least one therapeutic agent or medication for delivery to a subject. The dosage form comprises a given “formulation” and may be administered to a patient in the form of a lozenge, pill, tablet, capsule, membrane, strip, liquid, patch, film, gel, spray or other form. A dosage from comprising a formulation described herein may be used to deliver one or more tocotrienol that may be administered by the oral transmucosal route.

The term “subject” includes any subject, generally a mammal (e.g., human, canine, feline, equine, bovine, ungulate etc.), in which treatment with a tocotrienol is desired. In some embodiments, the subject is human, canine or equine.

The term “mucosal membrane” refers generally to any of the mucus-coated biological membranes in the body. Absorption through the mucosal membranes of the oral cavity is of particular interest. Thus, buccal, sublingual, gingival and palatal absorption are specifically contemplated by the present application.

The term “therapeutically effective amount” means an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

The term “T max” as used herein means the time point of maximum observed plasma concentration. The term “C.” as used herein means the maximum observed plasma concentration. The term “AUC” as used herein means “area under the curve” in a plot of concentration of drug in plasma versus time. AUC is usually given for the time interval zero to infinity, however, clearly plasma drug concentrations cannot be measured ‘to infinity’ for a patient so mathematical approaches are used to estimate the AUC from a limited number of concentration measurements. In a practical sense, the AUC (from zero to infinity) represents the total amount of drug absorbed by the body, irrespective of the rate of absorption. This is useful when trying to determine whether two formulations of the same dose release the same dose of drug to the body. The AUC can also be given for the time interval zero to the final measured time point (e.g. AUC₀₋₂₄ for a 24 hour experiment). The AUC of a transmucosal dosage form compared to that of the same dosage administered intravenously serves as the basis for a measurement of bioavailability. The term “Tₒₙₛₑₜ” as used herein means the observed “time of onset” and represents the time required for the plasma drug concentration to reach 50% of the maximum observed plasma concentration, Cₘₐₓ.

### Formulation

The present application provides a formulation for oral transmucosal administration of at least one tocotrienol or derivative thereof, comprising: a first composition comprising at least one tocotrienol or a derivative thereof, starch or a derivative of thereof, and silicon dioxide; and a second composition comprising one or more excipients, wherein the first composition and the second composition are combined to form the formulation.

PCT/AU2013/001310 describes a formulation for transmucosal administration comprising one or more tocotrienols. The present inventors have found the formulation described in PCT/AU2013/001310 tends to become sticky and leaches a viscous oily substance during certain steps in the manufacturing process, for example the step where a powder is pressed into a tablet, leading to fouling of the machinery used for high throughout manufacturing. The fouling requires frequent and thorough cleaning of the machinery for manufacturing and renders the process unsuitable for high throughput manufacturing which is required for commercial manufacture of a product. These formulations were prepared by combining a distillate concentrate of annatto seed (e.g. DeltaGold 70 supplied by American River Nutrition which is in the form of a viscous oil) with one or more excipients. The inventors found that the viscous oil was difficult to handle (e.g. difficult to aliquot accurately by volume or by weight) and to combine with other excipients and that when tablets were pressed using the formulation described in PCT/AU2013/001310, a viscous and sticky yellow oil leached out of the tablets fouling the machinery, particularly when the tablet presses heated up during manufacture. This occurred most frequently when tablets were being pressed during high throughput commercial—scale manufacturing (e.g. 10,000 to >100,000 tablets per day) and required that the tablet press was cleaned frequently which is impractical for commercial-scale manufacture. The present inventors also found that using the oil made it difficult to vary the key properties of the tablets which are required to deliver the tocotrienols transmucosally such as dissolution time. Accordingly, there is a need for an alternative formulation comprising tocotrienols that is suitable for transmucosal administration, and preferably is stable under manufacturing conditions (e.g. doesn't leach oil) and/or is suitable for high throughput manufacture.

The present inventors have found that combining one or more tocotrienols with silicon dioxide and starch or a derivative thereof to form a first composition before combining the first composition with the remaining excipients provides advantages for formulating, processing and/or manufacturing of the formulation, for example at large or commercial scale. The formulations described herein are less sticky, less likely to foul the machinery used for high throughout manufacturing and/or have improved powder flow. In some embodiments, the formulations described herein are not sticky and not likely to foul the machinery. These formulations are more amenable to high throughput manufacture of tablets (e.g. no leaching of oils and no sticky tablet presses which require frequent cleaning) and are more stable at temperatures reached during manufacture.

In some embodiments, the formulation is a free flowing powder. As used herein, the term “free flowing” refers to the ability of particulates to readily flow in response to shear forces, for example, those encountered during manufacturing or use of the formulation. The flow of a powder can be measured using methods known to the person skilled in the art. Suitable techniques are described in <1174> of the U.S> Pharmacopeia (available online) and titled “Powder Flow”. Examples include, angle of repose, flow through an orifice, shear cell methods and compressibility index and Hausner ratio. In some embodiments, free-flowing particles will have an angle of repose less than about 50°. In some embodiments, free-flowing particles will have an angle of repose less than about 40°. In some embodiments, free-flowing particles will have an angle of repose less than about 35°. In some embodiments, free-flowing particles will have a compressibility index of less than 25. In some embodiments, free-flowing particles will have a compressibility index of less than 20. In some embodiments, free-flowing particles will have a compressibility index of less than 15. In some embodiments, free-flowing particles will have a Hausner ratio of between 1.0 and 1.34. In some embodiments, free-flowing particles will have a Hausner ratio of between 1.0 and 1.25. In some embodiments, free-flowing particles will have a Hausner ratio of between 1.0 and 1.18. In some embodiments, the powder is sufficiently free flowing so that the formulation can be formed into a solid dosage form (e.g. pressed into a tablet, lozenge or wafer or filled into a capsule) without fouling of the manufacturing equipment.

The formulation describes herein comprises a first composition comprising (i) one or more tocotrienols, (ii) silicon dioxide and (iii) starch or a derivative thereof. The first composition is then combined with one or more excipients to form the formulation. Typically, the one or more excipients form a second composition.

a. Tocotrienol

The formulations described herein include one or more tocotrienols or a derivative thereof. The tocotrienols, or derivatives thereof, that can be used in the formulations, process, methods and uses of the present disclosure include naturally occurring tocotrienols (extracted from natural sources) and synthetic tocotrienols. Naturally occurring tocotrienols include alpha, beta, gamma and delta tocotrienols. While naturally occurring tocotrienols are known to exist in only one stereoisomeric form, other stereoisomers may be produced synthetically.

Derivatives of tocotrienols include, but are not limited to; esters, amides, phosphorylated, nitrosylated and succinate/seleno-succinate forms of tocotrienols. In some embodiments, derivatives of tocotrienols include derivatives of tocotrienols that enhance the therapeutic effect are also included, such as phosphorylated, nitrosylated and succinate/seleno-succinate forms. One example of a method of modifying tocotrienols can be found in Vraka et al “Synthesis and study of the cancer cell growth inhibitory properties of α-, γ-tocopheryl and γ-tocotrienyl 2-phenylselenyl succinates” Bioorganic & Medicinal Chemistry 14 (2006) 2684-2696, which is hereby incorporated by reference in its entirety.

The tocotrienols, or derivatives thereof, may be extracted from natural sources. For example, the tocotrienols may be derived from plant extracts such as palm oil, rice bran oil, wheat germ, barley and annatto bean. In some embodiments, the tocotrienols are derived from palm oil or annatto. In some embodiments, the tocotrienols are derived from annatto, for example the rainforest annatto plant (Bixa Orellana). In some embodiments, the tocotrienols are derived from annatto using the method described in U.S. Pat. No. 6,350,453, which is hereby incorporated by reference in its entirety.

The formulations described herein may include one form of tocotrienol, or derivative thereof, or a mixture of different tocotrienols, or derivatives thereof. In some embodiments, the one or more tocotrienols is selected from the group consisting of alpha tocotrienol, beta tocotrienol, gamma tocotrienol and delta tocotrienol and a combination thereof. In some embodiments, the one or more tocotrienols comprises gamma tocotrienol and/or delta tocotrienol. In some embodiments, the one or more tocotrienols consists of gamma tocotrienol and/or delta tocotrienol. In some embodiments, the one or more tocotrienols comprises delta tocotrienol.

In some embodiments, the formulation comprises gamma tocotrienol and delta tocotrienol, where the delta-to-gamma ratio of tocotrienols is between about 1:100 to 100:1. In some embodiments, the delta-to-gamma ratio of tocotrienols is between 1:25 to 25:1. In some embodiments, the delta-to-gamma ratio of tocotrienols is between 1:15 to 15:1. In some embodiments, the delta-to-gamma ratio of tocotrienols is between 1:1 to 12:1, for example 1:1, 2:1, 3:1, 4:1, 5:1, 6:1, 7:1. 8:1, 9:1, 10:1, 11:1 and 12:1. In some embodiments, the delta-to-gamma ratio of tocotrienols is between 5:1 and 12:1.

In some embodiments, the tocotrienols may be present in a tocotrienol-rich fraction produced from a plant extract. The tocotrienols in the formulation described herein may be isolated from other components of a plant extract, or may be present in combination with other plant components. In some embodiments, the tocotrienol-rich fraction may include some alpha tocopherol components. In some embodiments, the tocotrienol component in the formulation is greater than the tocopherol component. In some embodiments, the tocotrienol-rich fraction includes not more than about 50%, not more than about 40%, not more than about 30%, not more than about 20%, no more than about 10%, no more than about 5%, no more than about 2% alpha tocopherol or no more than about 1% tocopherol. In a some embodiments, the tocotrienol-rich fraction comprises less than about 1% tocopherol. In a some embodiments, the tocotrienol-rich fraction is substantially free of tocopherol (e.g. alpha-tocopherol).

In some embodiments, the tocopherol may be removed or modified such that the competitive activity with tocotrienols has been eliminated or reduced. A person skilled in the art would appreciate that there any number of means by which this could be achieved including but not limited to enzymatic modification (see Tones et al “Enzymatic Modification for Ascorbic Acid and Alpha-Tocopherol Enhances their Stability in Food and Nutritional Application” The Open Food Science Journal 2008, 2, 1-9). In some embodiments, the tocotrienol-rich fraction is derived from a natural source that is low in tocopherols.

The first composition described herein comprises one or more tocotrienols. The weight % of total tocotrienols present in the first composition (as a total weight % of the first composition) may typically be provided between 0.1 to 60, 1 to 50, 10 to 40, or 25 to 35. The weight % of total tocotrienols present in the first composition may be at least about 0.1, 1, 5, 10, 15, 20, 25, 30, 35, or 40. The weight % of total tocotrienols present in the first composition may be less than about 60, 55, 50, 45, 40, 35, or 30. In some embodiments, the weight % of total tocotrienols present in the first composition is between 20 to 40. In some embodiments, the weight % of total tocotrienols present in the first composition is between 25 to 35. In some embodiments, the weight % of total tocotrienols present in the first composition is between 30 to 33.6. In some embodiments, the weight % of total tocotrienols present in the first composition is between 36.4 to 38.5. The weight % of total tocotrienols present in the first composition may be provided in a range between any two of these upper and/or lower values.

In some embodiments, the one or more tocotrienols present in the first composition are derived from the annatto plant. In some embodiments, the first composition comprises an oil comprising one or more tocotrienols, such as DeltaGold 70 oil available from American River Nutrition. DeltaGold70 is an extract from annatto seed with 70% tocotrienol and contains approximately 90% delta-tocotrienol and 10% gamma-tocotrienol. As would be appreciated by the person skilled in the art the amount of extract present in the first composition is determined by the wt % of tocotrienol present in the extract. By way of example, where the extract is DeltaGold 70, the weight % of extract present in the first composition (as a total weight % of the first composition) may typically be provided between 0.14 to 86, 1.4 to 72, 14 to 58, or 35 to 50. The weight % of extract present in the first composition may be at least about 0.14, 1.4, 7, 15, 21.4, 28, 35, 43, 50, or 57. The weight % of total tocotrienols present in the first composition may be less than about 86, 79, 72, 64, 57, 50, or 43. In some embodiments, the weight % of extract present in the first composition is between 44 to 52. In some embodiments, the weight % of extract present in the first composition is between 45 to 52. In some embodiments, the weight % of extract present in the first composition is between 44 to 47. The weight % of total tocotrienols present in the first composition may be provided in a range between any two of these upper and/or lower values.

The formulations described herein comprises a therapeutically effective amount of the one or more tocotrienols. In some embodiments, the amount (in mg) of total tocotrienols present in the formulation may typically be provided between 10 to 200, 20 to 200, 20 to 180, 20 to 160, 20 to 140, 20 to 120, 20 to 100, 20 to 80, or 20 to 60. The amount (in mg) of total tocotrienols present in the formulation may be at least about 20, 25, 30, 35, 40, 45, 50, 55, 60, 65, 70, 75, 80, 85, 90, 95, 100, 110, 120, 130, 140, 150, 160, 170, 180, 190, 200. The amount (in mg) of total tocotrienols present in the formulation may be less than about 200, 180, 160, 140, 120, 100, 90, 80, 70, 60, 55, 50, 45, 40, 35, or 30. In some embodiments, the amount (in mg) of total tocotrienols present in the formulation is between about 20 to 200. In some embodiments, the amount (in mg) of total tocotrienols present in the formulation is between about 20 to 80. In some embodiments, the amount (in mg) of total tocotrienols present in the formulation is between about 20 to 60. In some embodiments, the amount (in mg) of total tocotrienols present in the formulation is about 20, 40 or 60. The amount (in mg) of total tocotrienols present in the formulation may be provided in a range between any two of these upper and/or lower values.

b. Starch or a Derivative Thereof

The first composition described herein comprises starch or a derivative thereof. As used herein, the term “starch” is given its ordinary meaning in the art. Starch derivatives (also called modified starch) may be prepared by physically, enzymatically, or chemically treating native starch, for example, using methods known to the person skilled in the art. In some examples, the starch derivative (is prepared by treating with acid, roasting it, treating with base (e.g. sodium hydroxide or potassium hydroxide), adding a positive charge, treating it with emulsifiers or treating it with starch ether. Examples of starch or derivatives thereof include, but are not limited to, waxy starches, modified starches, native starches and dextrins. In some embodiments, the starch or derivative thereof is a dextrin or a modified food starch. In some embodiments, the starch or derivative thereof is a modified food starch.

In some embodiments, the starch or derivative thereof is a dextrin. Dextrin is a starch derivative and is a low molecular weight carbohydrate that may be produced by the hydrolysis of starch or glycogen, for example, by heat, alkali and enzymes. The starch may be from maize, corn, tapioca, potato and the like. In some embodiments, the starch or derivative thereof is a tapioca dextrin.

Starch or derivatives thereof that are suitable for use in the formulations described herein may be derived from any suitable starch source. Suitable starch sources may, in some embodiments, include, but are not limited to, cereals, rice, wheat, maize, root vegetables, potatoes, corn, tapioca, cassava, acorns, arrowroot, arracacha, bananas, barley, breadfruit, buckwheat, canna, colacasia, katakuri, kudzu, malanga, millet, oats, oca, Polynesian arrowroot, sago, sorghum, sweet potatoes, rye, taro, chestnuts, water chestnuts, yams, beans, favas, lentils, mung beans, peas, chickpeas, and the like, and any combination thereof.

The weight % of the starch or derivative thereof (as a total weight % of the first composition) may typically be provided between 0.1 to 60, 10 to 60, 20 to 60, 30 to 60 or 40 to 60. The weight % of the starch or derivative thereof may be at least about 0.1, 1, 5, 10, 15, 20, 25, 30, 35, 40, 45, 50, or 55. The weight % of the starch or derivative thereof may be less than about 60, 55, 50, 45, 40, 35, 30, or 25. In some embodiments, the weight % of the starch or derivative thereof is between 40 to 60. In some embodiments, the weight % of the starch or derivative thereof is between 44 to 52. In some embodiments, the weight % of the starch or derivative thereof is between 44 to 47. In some embodiments, the weight % of the starch or derivative is between 45 to 52. The weight % of the starch or derivative thereof may be provided in a range between any two of these upper and/or lower values.

In one example, the first formulation comprises 45-52% (as a total weight % of the first composition) modified food starch. In another example, the first formulation comprises 44-47% (as a total weight % of the first composition) tapioca dextrin.

c. Silicon Dioxide

The first composition described herein also comprises silicon dioxide. Silicon dioxide may also be referred to as silica. The weight % of the silicon dioxide (as a total weight % of the first composition) may typically be provided between 0.001 to 10, 0.01 to 5, 0.1 to 2.5, or 1 to 2. The weight % of the silicon dioxide may be at least about 0.001, 0.01, 0.1, 1, 2, or 5. The weight % of the silicon dioxide may be less than about 10, 5, 4, 3, 2, or 1. In some embodiments, the weight % of the silicon dioxide is between 1 to 2. In some embodiments, the weight % of the silicon dioxide is about 1% (as a total weight % of the first composition). In some embodiments, the weight % of the silicon dioxide is about 1% (as a total weight % of the first composition). The weight % of the silicon dioxide may be provided in a range between any two of these upper and/or lower values.

d. One or More Excipients

The formulation described herein is formed by combining a first composition comprising at least one tocotrienol or a derivative thereof, starch or a derivative of thereof, and silicon dioxide; and a second composition comprising one or more excipients. The second composition can be formed prior to combining with the first composition (i.e. by combining two or more excipients prior to combining with the first composition) or can be formed “in situ” when combined with the first composition. For example, the one or more excipients may be combined to form the second composition when the formulation is formed. The present inventors have found that a formulation formed by combining one or more tocotrienols with starch or a derivative thereof and silicon dioxide before combining with the remaining excipients produces a formulation that is less sticky and/or less likely to foul manufacturing equipment.

Any excipient suitable for use in a formulation for transmucosal administration may be used. Suitable excipients include, but are not limited to bulking agents, binders, surfactants, bioadhesives/mucoadhesives, lubricants, disintegrants, stabilizers, solubilizers, glidants, diluents, flavours, sweeteners, and additives or factors that affect dissolution or disintegration time. Excipients are not limited to those above. Other suitable nontoxic pharmaceutically acceptable carriers for use in oral formulations can be found in Remington's Pharmaceutical Sciences, 17th Edition, 1985. As would be appreciated by the person skilled in the art, an excipient my fulfil more than one role in a formulation.

The formulations described herein may comprise at least one lubricant. Lubricants have several functions including preventing the adhesion of the tablets to the compression equipment and in some cases improving the flow of the granulation prior to compression or encapsulation. Non limiting examples of lubricants include, but are not limited to, stearic acid and divalent cations of such as magnesium Stearate, calcium stearate, etc., talc, glycerol monostearate and the like. In other embodiments, the lubricant is a water soluble lubricant. Non limiting examples of water soluble lubricants include, but are not limited to, boric acid, polyethylene glycol, sodium oleate, sodium benzoate, sodium acetate, sodium lauryl sulphate, and/or magnesium lauryl sulphate. The lubricant is typically present at 0.01-10% w/w, preferably between 1-5% w/w. In some embodiments, the water soluble lubricant comprises polyethylene glycol. In some embodiments, the polyethylene glycol is has a molecular weight between about 1,000 and 40,000. In some embodiments, the polyethylene glycol is has a molecular weight between about 4,000 and 10,000. In some embodiments, the polyethylene glycol is polyethylene glycol 4000, polyethylene glycol 6000 or polyethylene glycol 8000. In some embodiments, the polyethylene glycol is polyethylene glycol 8000. In some embodiments, the formulation comprises from about 1% about 5% by weight polyethylene glycol, for example, from about 1% about 2% by weight polyethylene glycol.

The formulations described herein may comprise a glidant. As used herein, the term “glidant” means a substance that, when added to a powder, improves the flowability of the powder, such as by reducing inter-particle friction. Exemplary glidants include but are not limited to silicas, silicon dioxide, fumed silica, CAB—O-SIL® M-5P, AEROSIL®, talc, starch, and magnesium aluminium silicates. In some embodiments, the glidant is silicon dioxide. In some embodiments, the silicon dioxide is SIPERNAT® 180 PQ supplied by Evonick Resource Efficiency GmbH. In some embodiments, the particle size (d50) of the silicon dioxide is between about 10 and 20 μm. In some embodiments, the particle size (d50) of the silicon dioxide is about 14 μm. Particle size can be measured using techniques known to the person skilled in the art, for example, laser diffraction following ISO 13320-1. In some embodiments, the formulation comprises from about 1% about 5% by weight glidant, for example, from about 1% about 2% by weight glidant. In embodiments where the glidant is silicon dioxide, the amount of glidant present in the formulation is separate to the amount of silicon dioxide present in the first composition.

The formulations described herein may comprise one or more binders. Binders facilitate binding of the excipients into a single dosage form. Exemplary binders are selected from the group consisting of cellulosic derivatives (such as methylcellulose, carboxymethyl cellulose, hydroxyethyl cellulose, hydroxy ethylmethyl cellulose, etc), starch derivatives (such as partially pregelatinized corn starch), polyacrylates (such as Carbopol, polycarbophil, etc), Povidone (all grades), Polyox of any molecular weight or grade, irradiated or not, starch, polyvinylpyrrolidone (PVP), Avicel, and the like. In some embodiments, the binder is partially pregelatinized corn starch (also referred to as Starch 1500®). The binder is typically present at 0.5-60% w/w, for example, 1-30% w/w or 1.5-15% w/w. In some embodiments, the formulation comprises from about 5% about 10% by weight partially pregelatinized corn starch, for example, from about 8% about 10% by weight partially pregelatinized corn starch. In embodiments where the binder is starch or a derivative thereof, the amount of binder present in the formulation is separate to the amount of starch or derivative thereof present in the first composition.

The formulations described herein may comprise one or more diluents, fillers or bulking agents. Non-limiting examples of suitable bulking agents include lactose USP, Starch 1500, mannitol, sorbitol, maltodextrin, malitol or other non-reducing Sugars; microcrystalline cellulose (e.g., Avicel), dibasic calcium phosphate (anhydrous or dihydrate). Sucrose, etc. and mixtures thereof. In some embodiments, the diluent is mannitol (e.g. mannitol Pearlitol 200SD). The use of mannitol provides a number of advantages. Without wishing to be bound by theory, it is thought the mannitol provides a pleasant taste and mouthfeel, lubricant insensitivity and/or ease of mixing. The diluent agent is typically present at 20-95% by weight, or 40-90% by weight, or 60-80% by weight, or 65-75% by weight.

The formulation described herein may also contain one or more flavours, sweeteners and/or colorants such as aspartame, lactose, sucrose, other artificial sweeteners; ferric oxides and FD&C lakes.

In some embodiments, the formulation comprises one or more flavours, for example peppermint flavour. Any suitable flavour may be used. In some embodiments, the formulation comprises peppermint flavour. While the amount of flavour included in the formulation will depend on the flavour, the typical amount of flavour present is between about 0.1-5% by weight, for example between 3-5% by weight.

In some embodiments, the formulation comprises one or more sweeteners. Any suitable sweetener may be used. In some embodiments, the sweetener is sucralose. While the amount of sweetener included in the formulation will depend on the sweetener, the typical amount of sweetener present is between about 0.01-1% by weight, for example between 0.1-0.5% by weight.

In some embodiments, the formulation does not comprise an amino acid. For example, in some embodiments, the formulation does not comprise arginine and/or leucine.

The formulation may also comprise one or more additives to help stabilize the tocotrienol from chemical of physical degradation. Such degradation reactions may include oxidation, hydrolysis, aggregation, deamidation, etc. Appropriate excipients that can stabilize the tocotrienols may include anti-oxidants, anti-hydrolytic agents, aggregation-blockers etc. Anti-oxidants may include BHT, BHA, vitamins, citric acid, EDTA, sodium bisulfate, sodium metabisulfate, thiourea, amino acids such as methionine, etc.

The formulations may comprise at least one bioadhesive (mucoadhesive) agent or a mixture of bioadhesives to promote adhesion to the oral mucosa during drug delivery. In addition, the bioadhesive agents may also be effective in controlling the dosage form erosion time and/or, the dissolution kinetics over time when the dosage form is wetted by saliva. In addition, some of the mucoadhesives may also serve as binders in the formulation to provide necessary bonding to the dosage form.

Exemplary mucoadhesive or bioadhesive materials, are selected from the group consisting of natural, synthetic or biological polymers, lipids, phospholipids, and the like. Examples of natural and/or synthetic polymers include cellulosic derivatives (such as methylcellulose, carboxymethyl cellulose, hydroxyethyl cellulose, hydroxyethylmethyl cellulose, microcrystalline cellulose, etc), natural gums (such as guar gum, Xanthan gum, locust bean gum, karayagum, veegum etc), polyacrylates (such as Carbopol, polycarbophil, etc), alginates, thiol-containing polymers, polyoxyethylenes, polyethylene glycols (PEG) of all molecular weights (preferably between 1000 and 40,000 Da, of any chemistry, linear or branched), dextrans of all molecular weights (preferably between 1000 and 40,000 Da of any source), block copolymers, such as those prepared by combinations of lactic & glycolic acid (PLA, PGA, PLGA of various viscosities, molecular weights and lactic-to-glycolic acid ratios) polyethylene glycol-polypropylene glycol block copolymers of any number and combination of repeating units (such as Pluronics, Tektronix or Genapol block copolymers), combination of the above copolymers either physically or chemically linked units (for example PEG-PLA or PEG-PLGA copolymers) mixtures. In some embodiments, the bioadhesive material is selected from the group of polyethylene glycols, polyoxyethylenes, polyacrylic acid polymers, such as Carbopols (such as Carbopol 71G, 934P. 971 P974P) and polycarbophils (such as Noveon AA-1, Noveon CA-1, Noveon CA-2), cellulose and its derivatives. In some embodiments, it is polyethylene glycol, Carbopol, and/or a cellulosic derivative or a combination thereof. A formulation may contain one or more different bioadhesives in any combination. In some embodiments, the mucoadhesive/bioadhesive excipient is polyethylene glycol, for example polyethylene glycol having a molecular weight between 1,000 and 40,000 Da, or between 4,000 and 10,000 Da, for example polyethylene 8000. In some embodiments, the mucoadhesive/bioadhesive excipient is typically present at 1-50% by weight, or 1-40% by weight or 1-30% by weight, or 1-20% by weight, or 1-10% by weight or 1-5% by weight or 1-2% by weight.

In some embodiments, the excipient comprises polyethylene glycol (referred to collectively herein as PEG). In some embodiments, the polyethylene glycol has a molecular weight average from about 1,000 to 40,000 Daltons. In some embodiments, the polyethylene glycol has a molecular weight average from about 4,000 to 20,000 Daltons. In some embodiments, the polyethylene glycol has a molecular weight average from about 6,000 to 10,000 Daltons. In some embodiments, the polyethylene glycol is polyethylene glycol 8000 (PEG 8000). In some embodiments, the formulation comprises from about 1% about 5% by weight polyethylene glycol, for example, from about 1% about 2% by weight polyethylene glycol.

In some embodiments, the one or more excipients form a second composition prior to combining with the first composition. In some embodiments, the one or more excipients form a second composition at the same time as combining with the first composition.

e. Other Actives

In some embodiments, the formulation may comprise or be administered with any other compound that will complement and enhance the therapeutic effect of the tocotrienol, or derivative thereof, including, but not limited to, monoglycerides, lignans isoprenoids, amino acids, CoQ10, polyphenols, omega-3 fatty acids, endocannabinoid system agonists and antagonists, flavonoids, carotenoids, mono and oligosaccharides, niacin and bioactive peptides. In some embodiments, the formulation may comprise or be administered with extracts from sesame seeds and or sesame lignans.

The formulation described herein comprises a first composition and a second composition comprising one or more excipients. In some embodiments, the ratio of the weight of the first composition to the second composition (for the avoidance of doubt, the combined weight of each excipient that does not form part of the first composition) is between about 1:1 and 1:50, between about 1:2 and 1:20, between about 1:5 and 1:10. In some embodiments, the weight ratio of the first composition to total weight of excipients is about 1:1, 1:2, 1:3, 1:4, 1:5, 1:6, 1:7, 1:8, 1:9, 1:10, 1:11, 1:12, 1:13, 1:14 or 1:15.

The formulation described herein provides one or more advantages over one or more existing formulations. For example, in some embodiments, the formulation has a good mouth feel, is palatable, has an agreeable aroma and/or has a suitable dissolution time to promote transmucosal delivery. In some embodiments, the formulation is suitable for high throughput manufacture of tablets and other formats such as wafers and lozenges due to no oil leaching out during the pressing process which converts the free-flowing powder into a solid form such as a tablet, a wafer or a lozenge.

In some embodiments, the formulation comprises or consists of: a first composition comprising one or more tocotrienol, silicon dioxide, and a dextrin; and a second composition comprising polyethylene glycol, partially pregelatinized corn starch, silicon dioxide, mannitol, a flavour and a sweetener. In some embodiments, the formulation comprises or consists of: a first composition comprising one or more tocotrienol, silicon dioxide, and a dextrin; and a second composition comprising polyethylene glycol 8000, silicon dioxide, partially pregelatinized corn starch, mannitol, a flavour and a sweetener.

In some embodiments, the formulation comprises or consists of one or more tocotrienol, silicon dioxide, dextrin, polyethylene glycol, partially pregelatinized corn starch, mannitol, a flavour and a sweetener. In some embodiments, the formulation comprises or consists of one or more tocotrienol, silicon dioxide, dextrin, polyethylene glycol 8000, partially pregelatinized corn starch, mannitol, a flavour and a sweetener.

### Oral Transmucosal Drug Delivery Dosage Forms

To the best of the inventors' knowledge, to date, all commercially available products containing tocotrienols are for oral ingestion. The orally ingested formulations are often in the form of capsules that comprise an oil or self-emulsifying drug delivery system. The formulations describe herein are suitable for transmucosal administration, for example, the formulations have a flavour, aroma, mouth feel, dissolution time and/or pharmacokinetics that are amenable to transmucosal delivery of tocotrienols.

As used herein, the term “transmucosal” administration and the like is meant to encompass all forms of delivery across or through a mucosal membrane. This can include nasal, sublingual, vaginal or rectal, or ocular routes. In particular, “oral transmucosal” administration includes delivery across the mucosal membranes of the oral cavity, for example, the sublingual, gingival, buccal and palatal mucosal tissues. In some embodiments, the formulation is suitable for oral transmucosal administration. In some embodiments, the formulation is suitable for sublingual, buccal and/or mucosal transmucosal administration. In some embodiments, the formulation is suitable for sublingual and/or buccal transmucosal administration.

As used herein, the term “sublingual”, means literally “under the tongue” and refers to a method of administering substances via the mouth in such a way that the substances are rapidly absorbed via the blood vessels under the tongue rather than via the digestive tract. Among the various transmucosal sites, the mucosa of the sublingual cavity is found to be the most convenient and easily accessible site for the delivery of therapeutic agents for both local and systemic delivery as sustained release dosage forms because it of its abundant vascularization. Direct access to the systemic circulation through the internal jugular vein bypasses the hepatic first pass metabolism leading to high bioavailability. Further, owing to the highly vascularized nature of the sublingual mucosal membrane and the reduced number of epithelial cell layers compared to other mucosal membranes, absorption of therapeutic substances occurs rapidly, thus allowing for direct access to the systemic circulation and thus enable quick onset of action while avoiding complications of oral administration.

The dosage form is typically a “sublingual dosage form”, but in some cases other oral transmucosal routes may be employed. The dosage form is a substantially homogeneous composition which comprises one or more tocotrienols, starch or a derivative thereof, silicon dioxide and one or more excipients. The dosage forms of the disclosure are adapted for oral transmucosal (for example sublingual) delivery of a one or more tocotrienols and typically have a dissolution time that optimises transmucosal absorption (i.e. the dosage form does not dissolve too fast or too slow, both of which are expected to maximise oral ingestion compared to transmucosal absorption). In some examples, the dissolution time is from 5 seconds up to a time selected from, 10 seconds, 15 seconds, 30 seconds, 45 seconds, 1 minute or 2 minutes. In some embodiments, the formulation has dissolved within about 30 seconds, within 25 seconds, within 20 seconds, within 15 seconds, or within 10 seconds. In some embodiments, the formulation has dissolved within 10 to 30 seconds. In some embodiments, the formulation has dissolved within 15 to 30 seconds.

Without wishing to be bound by theory, oral transmucosal delivery of tocotrienols, or derivatives thereof, as described herein is believed to have a number of advantages compared to traditional oral delivery. Firstly, it may overcome the problem of low intestinal absorption of tocotrienols by ensuring high absorption via the sublingual lymphatic system. This technique will also bypass the low affinity of tocotrienols for the alpha-tocopherol transport protein, because the lymphatic circulation will deliver them directly to the target tissues, without the necessity for incorporation by the liver into triglyceride, and lipoproteins and export into the circulation. This method will also minimise first-pass metabolism of tocotrienols by the liver and increase the amount of tocotrienols delivered to other organs. Furthermore, as demonstrated in PCT/AU2013/001310, the inventors of the present application have shown a surprising increase in efficacy of a sublingual formulation of tocotrienol over an oral formulation in reducing Delayed Onset Muscle Soreness (DOMS), improving muscle recovery after exercise, maintenance of peak muscle power and exercise endurance in humans. Finally, transmucosal administration of the formulation described herein is thought to provide improved bioavailability when administered in the fasted state.

Oral transmucosal drug delivery is simple, non-invasive, and can be administered with minimal discomfort which is expected to promote compliance by patients and consumers. This, in turn, is critically important for indications which are chronic where patients are required to self-administer the drug over an extended period of time (e.g. many years). Generally, oral transmucosal delivery of pharmaceuticals is achieved using dosage forms such as lozenges or tablets, however, liquids, sprays, gels, gums, powders, and films may also be used. In some embodiments, the dosage form is a solid dosage form. In some embodiments, the solid dosage form is a tablet, lozenge, wafer, a pill, a capsule, a membrane, a strip, a patch, a film, or a powder. In some embodiments, solid dosage form is a powder or a tablet. In some embodiments, solid dosage form is a powder. In some embodiments, solid dosage form is a tablet.

### Process

The present inventors have found that by combining one or more tocotrienols with a starch or derivative thereof (such as dextrin) and silicon dioxide to form a first composition prior to combining with one or more excipients provides advantages for formulating, processing and/or manufacturing the formulation.

In some embodiments, the process of making a formulation described herein comprises the steps of weighing individual ingredient, combining one or more tocotrienol (e.g. DeltaGold 70 oil), starch or derivative thereof, and silicon dioxide to form a powder, combining the powder with one or more excipients and mixing to form a powder (e.g. a free flowing powder). In some embodiments, combining the first composition with one or more excipients comprises mixing or agitation to form a homogeneous and free flowing powder. In some embodiments, the mixing step comprises rotating and/or agitating the ingredients in a mixer (e.g. a v-mixer) for a fixed and at a speed which is appropriate to create a well-mixed (e.g. homogeneous) free-flowing powder. The time and speed can be determined by the person skilled in the art and should not be too slow so that the individual ingredients do not mix properly (i.e. potentially resulting in a mixture which is not homogeneous) and not too fast so that the ingredients disintegrate. In some embodiments, the ingredients are mixed for about 10 to 60 minutes, or about 20 to 40 minutes, or about 20 to 30 minutes, or about 25 minutes. In some embodiments, the ingredients are mixed at a speed of about 10 rpm (revolutions per minute) to 50 rpm, or about 20 to 40 rpm, or about 25 to 35 rpm. In some embodiments, the mixing step comprises mixing in a rotator at speeds in the range of 10-50 revolutions per minute for 10-45 minutes to give a homogeneous, free-flowing powder. Other processes for preparing a formulation for oral transmucosal administration of at least one tocotrienol or derivative thereof known to the person skilled in the art may be used provided the first composition comprising one or more tocotrienols, starch or a derivative thereof and silicon dioxide is formed before mixing with other excipients.

In some embodiments, the first composition can be obtained from a commercial supplier, for example, DeltaGold 30 or DeltaGold 35 available from American River Nutrition (Hadley, MA, USA). In alternative embodiments, the first composition can be prepared by combining a suitable plant extract comprising one or more tocotrienols (e.g. DeltaGold 70 oil) with starch or a derivative thereof and silicon dioxide. DeltaGold 70 oil is also available from American River Nutrition (Hadley, MA, USA).

The process produces a powder formulation, for example a free flowing powder formulation. The powder formulation may be used for transmucosal administration of one or more tocotrienols. In some embodiments, the powder can be packed into sachets or stick packs (sachets which are longer than they are wide).

In some embodiments, the powder formulation is further processed to form a dosage form suitable for transmucosal delivery. It will be understood that the formulation will be converted into a dosage form suitable for transmucosal delivery to a subject using procedures routinely employed by those of skill in the art. Many methods of making dosage forms for use in the methods and uses described herein are known in the art and may be employed in practicing the present disclosure, such as direct compression, etc. For example, the powder may be pressed into a tablet or filled into a capsule.

The process described herein is suitable for commercial manufacture of the disclosed formulation. In some embodiments, the process described herein is suitable for large scale manufacture of the disclosed formulation. For example, in some embodiments the process is suitable for the manufacture of at least 1,000 units per day, at least 5,000 units per day, at least 10,000 units per day, at least 20,000 units per day, or at least 50,000 units per day. In some embodiments the process is suitable for the manufacture of between 10,000 to 200,000 units per day.

### Uses

The formulations described herein may be used for the treatment of any disease or condition that is capable of treatment with a tocotrienol. Accordingly, the present disclosure provides a method of treating or preventing a disease or condition amenable to treatment with a tocotrienol comprising transmucosal administration of the formulation described herein. In some embodiments, the disease or condition is selected from the group consisting of post exercise muscle soreness, delayed onset muscle soreness, muscle recovery after exercise, maintenance of peak muscle power, fibrosis, hypertension, inflammation, stroke, cancer, elevated cholesterol and/or triglycerides, baldness, hypertrophy and a condition resulting from radiation exposure.

Tocotrienols have been shown to improve control of blood glucose and insulin response (Kuhad et al (2009) “Suppression of NF-kappa beta signalling pathway by tocotrienol can prevent diabetes associated cognitive deficits” Pharmacol. Biochem. Behav. 92, 251-259). Accordingly, the present application also provides a method of stabilizing and/or controlling blood glucose levels in a subject comprising transmucosal administration of the formulation as described herein. In some embodiments, there is provided a method of stabilizing and/or controlling blood glucose levels in a subject comprising transmucosal administration of a formulation described herein, wherein the formulation is suitable for buccal, sub-lingual, or mucosal administration, though preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptides, that will complement and enhance the effect of tocotrienols with respect to stabilizing and/or controlling blood glucose levels.

The present application also provides a methods of improving exercise endurance in a subject comprising transmucosal administration of the formulation as described herein. In some embodiments, there is provided a method of improving exercise endurance in a subject comprising transmucosal administration of a formulation described herein, wherein the formulation is suitable for buccal, sub-lingual, or mucosal administration, preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptides, that will complement and enhance the effect of tocotrienols with respect to improvement in exercise endurance.

The present application also provides a method of promoting weight loss in a subject comprising transmucosal administration of the formulation as described herein. In some embodiments, there is provided a method of promoting weight loss in a subject comprising transmucosal administration of a formulation described herein, wherein the formulation is suitable for buccal, sub-lingual, or mucosal administration, preferably sub-lingual and/or buccal administration. In some embodiments, the formulation is administered in combination with a compound, such as monoglycerides, lignans isoprenoids, polyphenols, flavonoids, carotenoids, mono and oligosaccharides, niacin and bioactive peptides, that will complement and enhance the effect of tocotrienols with respect to promoting weight loss.

The present application also provides a method of reducing hypertension in a subject comprising transmucosal administration of the formulation as described herein. In some embodiments, there is provided a method of reducing hypertension in a subject comprising transmucosal administration of a formulation described herein, wherein the formulation is suitable for buccal, sub-lingual or mucosal administration, preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptides, that will complement and enhance the effect of tocotrienols with respect to reducing hypertension.

The present application also provides a method of treating ischemic disease in a subject comprising transmucosal administration of the formulation as described herein. In some embodiments, there is provided a method of treating ischemic disease in a subject comprising transmucosal administration of a formulation described herein, wherein the formulation is suitable for buccal, sub-lingual or mucosal administration, preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to treating ischemic disease.

The present application also provides a method of reducing cholesterol and/or triglycerides in in a subject comprising transmucosal administration of the formulation as described herein. In some embodiments, there is provided a method of reducing cholesterol and/or triglycerides in a subject comprising administration of tocotrienol (and/or its derivatives), wherein the formulation is suitable for buccal, sub-lingual or mucosal administration, preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing cholesterol and/or triglycerides.

The present application also provides a methods of treating cancer in a subject comprising transmucosal administration of the formulation as described herein. In some embodiments, there is provided a method of treating cancer in a subject comprising transmucosal administration of tocotrienol (and/or its derivatives), wherein the formulation is suitable for buccal, sub-lingual or mucosal administration, preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to treating cancer.

The present application also provides a method of increasing the bioavailability of tocotrienols administered to a subject comprising transmucosal administration of the formulation as described herein. In some embodiments, there is provided a method of increasing the bioavailability of tocotrienols administered to a subject comprising transmucosal administration of tocotrienol (and/or its derivatives), wherein the formulation is suitable for buccal, sub-lingual or mucosal administration, preferably sub-lingual administration. In some embodiments, the formulation is administered in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to increasing the bioavailability of tocotrienols administered to animals.

The present application also provides a method of minimizing the dosage required to achieve a therapeutic effect in a subject comprising transmucosal administration of the formulation as described herein. In some embodiments, there is provided a method of minimizing the dosage required to achieve a therapeutic effect in a subject, comprising transmucosal administration of a formulation described herein, wherein the formulation is suitable for buccal, sub-lingual or mucosal administration, though preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to minimizing the dosage required to achieve a therapeutic effect by the administration of tocotrienols.

The present application also provides a method of reducing and/or inhibiting inflammation comprising transmucosal administration of the formulation as described herein. In some embodiments, there is provided a method of reducing and/or inhibiting inflammation by the transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing and/or inhibiting inflammation by the administration of tocotrienols.

The present application also provides a method of reducing and/or inhibiting post exercise muscle soreness comprising transmucosal administration of the formulation as described herein. In some embodiments, there is provided a method of reducing and/or inhibiting post exercise muscle soreness comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing and/or inhibiting post exercise muscle soreness by the administration of tocotrienols.

The present application also provides a method of reducing and/or inhibiting delayed onset muscle soreness comprising transmucosal administration of the formulation as described herein. In some embodiments, there is provided a method of reducing and/or inhibiting delayed onset muscle soreness comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing and/or inhibiting post exercise muscle soreness by the administration of tocotrienols.

The present application also provides a method of improving muscle recovery after exercise comprising transmucosal administration of the formulation as described herein. In some embodiments, there is provided a method of reducing and/or inhibiting delayed onset muscle soreness comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to improving muscle recovery after exercise by the administration of tocotrienols.

The present application also provides a method of improving the maintenance of peak muscle power comprising transmucosal administration of the formulation as described herein. In some embodiments, there is provided a method of reducing and/or inhibiting delayed onset muscle soreness comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to improving the maintenance of peak muscle power by the administration of tocotrienols.

The present application also provides a method of reducing and/or treating fibrosis comprising transmucosal administration of the formulation as described herein. In some embodiments, there is provided a method a method of reducing and/or treating fibrosis comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing and/or treating cardiac by the administration of tocotrienols. In some embodiments, the fibrosis is liver fibrosis or cardiac fibrosis.

The present application also provides a method of reducing and/or treating cardiac fibrosis comprising transmucosal administration of the formulation as described herein. In some embodiments, there is provided a method a method of reducing and/or treating cardiac fibrosis comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing and/or treating cardiac fibrosis by the administration of tocotrienols.

The present application also provides a method of reducing and/or treating liver fibrosis comprising transmucosal administration of the formulation as described herein. In some embodiments, there is provided a method a method of reducing and/or treating liver fibrosis comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to reducing and/or treating liver fibrosis by the administration of tocotrienols.

The present application also provides a method of treating radiation exposure in a subject comprising transmucosal administration of the formulation as described herein. In some embodiments, there is provided a method of treating radiation exposure in a subject comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to treating radiation exposure in an animal by the administration of tocotrienols.

The present application also provides a method of treating male pattern baldness comprising transmucosal administration of the formulation as described herein. In some embodiments, there is provided a method of treating male pattern baldness comprising transmucosal administration of a formulation described herein to a subject, wherein the formulation is suitable for buccal, sub-lingual or mucosal administration, preferably sub-lingual administration. In some embodiments, the formulation is used in combination with a compound, such as monoglycerides, lignans isoprenoids, polyphenols, flavonoids, carotenoids, mono and oligosaccharides, niacin and bioactive peptide, that will complement and enhance the effect of tocotrienols with respect to treating male pattern baldness by the administration of tocotrienols.

In some embodiments, the methods comprise transmucosal administration of the formulation as described herein. In some embodiments, the transmucosal administration is sublingual and/or buccal administration.

The formulations described herein may be used for the treatment of any disease or condition that is capable of treatment with a tocotrienol. Accordingly, the present disclosure provides a formulation as described herein for use in treating or preventing a disease or condition amenable to treatment with a tocotrienol comprising transmucosal administration of the formulation described herein. The present disclosure also provides use of a formulation as described herein for treating or preventing a disease or condition amenable to treatment with a tocotrienol comprising transmucosal administration of the formulation described herein. The present application also provides use of the formulation as described herein for the manufacture of a medicament for the treatment of a disease or condition amenable to treatment with a tocotrienol, wherein the formulation is formulated for transmucosal administration. In some embodiments, the disease or condition is selected from the group consisting of post exercise muscle soreness, delayed onset muscle soreness, muscle recovery after exercise, maintenance of peak muscle power, fibrosis, hypertension, inflammation, stroke, cancer, elevated cholesterol and/or triglycerides; baldness, hypertrophy and a condition resulting from radiation exposure.

The formulation described herein is for transmucosal administration. In some embodiments, the formulation is administered in the form of a pharmaceutical composition suitable for buccal, sub-lingual or mucosal administration, though preferably sublingual and/or buccal administration.

In some embodiments, the formulation is administered in combination with a further compound designed to complement and enhance the therapeutic effect of the tocotrienol, or derivative thereof, selected from the group consisting of: monoglycerides, lignans, isoprenoids, polyphenols, flavonoids, carotenoids, mono and oligosaccharides, niacin and bioactive peptides.

In some embodiments, the formulation described herein may be administered to a subject under fed conditions or fasted conditions. It is well understood in the art that the pharmacokinetic performance of some formulations is affected by the presence or absence of food in the gastro-intestinal system. As used herein, the term “fasted state” means that the subject has not ingested food for at least eight hours before the formulation described herein is administered. As used herein, the term “fed state” refers to a human who has eaten a United States Food and Drug Administration (FDA) standard high fat breakfast (or other meal containing a comparable quantity of fat and calories) within said time period. The meal is high in both fat (approximately 50% of total calorie content of the meal) and calories (approximately 800-1000 calories). In some embodiments, the formulation is administered to a subject in the fasted state.

A study by Yap et al (Pharmacokinetics and bioavailability of alpha-, gamma- and delta-tocotrienols under different food status. J Pharm Pharmacol. 2001 January; 53(1):67-71) found that the bioavailability of orally administered tocotrienols was increased when administered in the fed state, after a high fat meal. Without wishing to be bound by theory, it is thought that transmucosal administration of tocotrienols increases the bioavailability of tocotrienols in the fasted state. Accordingly, in some embodiments, the formulation is administered to a subject in the fasted state. This has the advantage of the subject not eating a high fat meal prior to administration which is often not a clinically relevant situation (i.e. most indications treated by tocotrienols require patients to abstain from high fat meals, e.g., patients with hyperlipidaemia or cancer should not consume high fat meals).

In some embodiments, the formulation described herein is administered at 20 mg/day, 40 mg/day, 60 mg/day, 80 mg/day, 100 mg/day, 120 mg/day, 140 mg/day, 160 mg/day, 180 mg/day, 200 mg/day, 300 mg/day or 400 mg/day. In some embodiments, the formulation described herein is administered at 20 mg/day, 40 mg/day, 60 mg/day, or 80 mg/day, or 120 mg/day. The formulation may be administered once, twice, three or four times per day. In some embodiments, the formulation described herein is administered at a dose of between 10 to 200, 20 to 200, 20 to 180, 20 to 160, 20 to 140, 20 to 120, 20 to 100, 20 to 80, or 20 to 60 mg tocotrienol at least once a day. The amount (in mg) of total tocotrienols administered per day may be at least about 20, 25, 30, 35, 40, 45, 50, 55, 60, 65, 70, 75, 80, 85, 90, 95, 100, 110, 120, 130, 140, 150, 160, 170, 180, 190, 200. The amount (in mg) of total tocotrienols administered per day may be less than about 200, 180, 160, 140, 120, 100, 90, 80, 70, 60, 55, 50, 45, 40, 35, or 30. In some embodiments, the amount (in mg) of total tocotrienols administered per day is between about 20 to 200. In some embodiments, the amount (in mg) of total administered per day is between about 20 to 80. In some embodiments, the amount (in mg) of total tocotrienols administered per day is between about 20 to 60. In some embodiments, the amount (in mg) of total tocotrienols administered per day is 20, 40, 60 or 80.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The subject matter of the present application is further described with reference to the following non-limiting examples.

### Example 1

The formulations exemplified herein are prepared according to the following method:
Upon delivery of the active material (e.g. DeltaGold 35 or DeltaGold30 powder) a sample is taken and analysed to assess compliance with product specifications.
On confirmation that the starting material is compliant with its product specifications, each individual ingredient, flavour and/or sweetener is weighed out using a scale.
Each ingredient is then placed into a mixer (e.g. a small v-mixer).
The mixture is rotated or agitated in the mixer for 25 minutes at a speed which is appropriate to create a well-mixed free-flowing powder (i.e. not too slow so that the individual ingredients do not mix properly and not too fast so that the ingredients disintegrate, e.g. 25-35 rpm).
On formation of a free flowing powder, a sample of the product is retained for future reference.
A sample is aliquoted out of the retained sample and examined for appearance, texture, and taste to make sure it meets the required specifications for the product.
If the sample is determined to comply with specifications, the powder can be packed into sachets or stick packs (sachets which are longer than they are wide) or further processed by pressing the powder into a tablet, lozenge, wafer or some other solid form which dissolves on contact with saliva.

### Example 2

| | ACTIVE | | |
|---|---|---|---|
| INGREDIENT | (MG) | MG/UNIT | WT % |
| Vitamin E Tocotrienols 35% DeltaGold | 40 | 114.286 | 16.8 |
| 35P | | | |
| Polyethylene Glycol 8000 Powder | 10 | 10 | 1.5 |
| Silicon Dioxide SIPERNAT 180 PQ | 12 | 12 | 1.8 |
| Starch 1500 (partially pregelatinized | 60 | 60 | 8.8 |
| corn starch) | | | |
| Mannitol Pearlitol 200 SD | 460 | 460 | 67.5 |
| Peppermint Flavour Powder | 25 | 25 | 3.7 |
| Sucralose | 0.7 | 0.7 | 0.1 |

### Example 3

| | ACTIVE | | |
|---|---|---|---|
| INGREDIENT | (MG) | MG/UNIT | WT % |
| Vitamin E Tocotrienols 35% DeltaGold | 60 | 171.429 | 16.8 |
| 35P | | | |
| Polyethylene Glycol 8000 Powder | 15 | 15 | 1.5 |
| Silicon Dioxide SIPERNAT 180 PQ | 18 | 18 | 1.8 |
| Starch 1500 (partially pregelatinized | 90 | 90 | 8.8 |
| corn starch) | | | |
| Mannitol Pearlitol 200 SD | 690 | 690 | 67.5 |
| Peppermint Flavour Powder | 37.5 | 37.5 | 3.7 |
| Sucralose | 1.05 | 1.05 | 0.1 |

### Example 4

| | ACTIVE | | |
|---|---|---|---|
| INGREDIENT | (MG) | MG/UNIT | WT % |
| Vitamin E Tocotrienols 35% DeltaGold | 20 | 57.143 | 9.1 |
| 35P | | | |
| Polyethylene Glycol 8000 Powder | 10 | 10 | 1.6 |
| Silicon Dioxide SIPERNAT 180 PQ | 12 | 12 | 1.9 |
| Starch 1500 (partially pregelatinized | 60 | 60 | 9.6 |
| corn starch) | | | |
| Mannitol Pearlitol 200 SD | 460 | 460 | 73.6 |
| Peppermint Flavour Powder | 25 | 25 | 4.0 |
| Sucralose | 0.7 | 0.7 | 0.1 |

### Example 5

The pharmacokinetics in rats of sublingually administered tocotrienols were assessed. The compositions for sublingual administration were prepared as described in examples 1, 2 and 4. The example 2 formulation was used to administer a dose of 1 mg/kg and the example 4 formulation was used to administer a dose of 3 mg/kg and 6 mg/kg. The composition was a powder.

The composition was administered sublingually to provide a dose of 1, 3 and 6 mg/kg tocotrienol. Male Sprague-Dawley rats (n=3 per group) were used in the study. The day before dosing, the rats were anaesthetised and the carotid artery was cannulated. The rats were then allowed to regain consciousness and fasted overnight with free access to water. On the day of dosing the rats were anaesthetised and positioned so that sublingual administration was possible and the rat was maintained in an upright position (bottom chin and belly facing the floor). The composition was placed under the tongue. Animals had cannulas implanted into the carotid artery under anaesthesia and cannulas exteriorized to facilitate sampling from freely moving conscious animals. Blood samples of 200 μl were collected for pharmacokinetic analysis at 0 (prior to dosing, baseline), 0.25, 0.5, 1, 1.5, 2, 3, 4, 6, 8 and 24 hours. Samples were centrifuged to provide 100 μl plasma samples and frozen for storage before analysis.

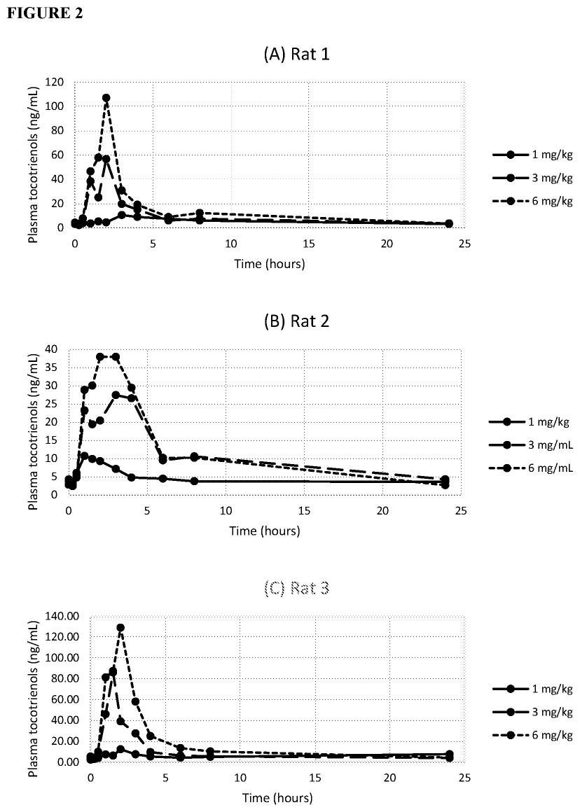

Plasma samples were analysed for delta tocotrienols using known techniques and the following pharmacokinetic descriptors were measured: Peak plasma concentration (Cₘₐₓ); time to peak plasma concentration (Tₘₐₓ); elimination half-life (T_{1/2}); area under the concentration time curve from time 0 to last time point evaluated (AUC₀₋ₜ); and area under the concentration time curve from time 0 and extrapolated to infinity. The plasma tocotrienol levels over 24 hours are presented in FIG. 2. As shown in FIG. 2 tocotrienols appeared rapidly in plasma, with maximum levels obtained within 2 hours of administration. The pharmacokinetic data are presented in Table 1.

TABLE 1
Rat Pharmacokinetic Descriptors for exemplified composition
administered sublingually at a dose of 1, 3 and 6 mg/kg.

| Rat No | Dose | AUC_{0-24 h} | Cₘₐₓ | Tₘₐₓ | T_{1/2} |
|---|---|---|---|---|---|
| Rat 1 | 1 mg/kg | 130.003 | 10.6 | 3.0 | 9.95 |
| Rat 1 | 3 mg/kg | 228.713 | 56.6 | 2.0 | 4.98 |
| Rat 1 | 6 mg/kg | 352.524 | 107.0 | 2.0 | 3.97 |
| Rat 2 | 1 mg/kg | 106.545 | 10.7 | 1.0 | 9.52 |
| Rat 2 | 3 mg/kg | 255.558 | 27.4 | 3.0 | 7.15 |
| Rat 2 | 6 mg/kg | 276.496 | 37.9 | 3.0 | 5.25 |
| Rat 3 | 1 mg/kg | 146.624 | 12.2 | 2.0 | 12.21 |
| Rat 3 | 3 mg/kg | 234.244 | 86.0 | 1.5 | 3.17 |
| Rat 3 | 6 mg/kg | 433.531 | 129.0 | 2.0 | 3.04 |

Compared to previous results observed for oral administration of a formulation of tocotrienols, the present formulation has a much longer elimination half-life (ranging from 3 to 12 hours compared to 2 to 7 hours for the previous oral formulation) suggesting a slower release of the drug.

### Example 6

The human pharmacokinetics of sublingually administered tocotrienols were assessed. The compositions for sublingual administration were prepared as described in example 1 and 2. The composition was a powder.

After consenting, the subjects were sublingually administered the composition of Example 2. The subject was sublingually administered 40 mg or 80 mg (2×40 mg) of total tocotrienol. Blood samples were collected from a forearm venous catheter before administration and at 5, 10, 15, 30, 45 minutes and 1, 1.5, 2, 3, 4, 6, and 8 hours post ingestion. A 4 ml blood sample was collected at each time point using a lavender Vacutainer (BD Product #367861) and the sample mixed with EDTA anticoagulant before being centrifuged (10 minutes, 2000 g). to provide plasma samples. Plasma samples were frozen until analysis.

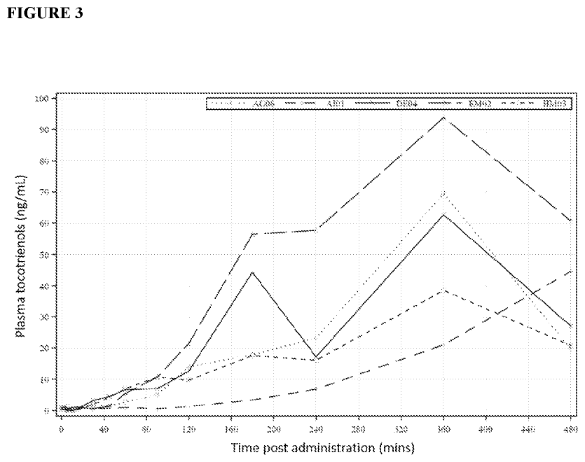

Plasma samples were analysed for tocotrienols using known techniques and the following pharmacokinetic descriptors were measured: Peak plasma concentration (Cₘₐₓ); time to peak plasma concentration (Tₘₐₓ); elimination half-life (T_{1/2}); area under the concentration time curve from time 0 to last time point evaluated (AUC₀₋ₜ). The plasma tocotrienol levels over 8 hours are presented in FIG. 3. The pharmacokinetic data are presented in Table 2.

TABLE 2
Human Pharmacokinetic Descriptors for exemplified
formulation administered sublingually.

| Sample | Dose | AUC | Cₘₐₓ | Tₘₐₓ | T_{1/2} |
|---|---|---|---|---|---|
| AG06 | 40 mg | 13550.08 | 69.4 | 360 | 329.1 |
| AJ01 | 40 mg | 6134.94 | 44.5 | 480 | 386.3 |
| DE04 | 40 mg | 14375.90 | 62.6 | 360 | 314.5 |
| HM03 | 40 mg | 9387.57 | 38.5 | 360 | 318.2 |
| EM02 | 80 mg | 24925.98 | 93.8 | 360 | 317.8 |

Both the Cmax and AUC increased linearly from the 40 mg dose to the 80 mg dose.

### Example 7

Selected dosage forms comprising the formulations described herein will be tested in a suitable animal model to evaluate the pharmacokinetics following sublingual administration. Comparisons of oral transmucosal drug delivery using formulations described herein relative to orally ingested tocotrienols as well as the formulations described in PCT/AU2013/001310 (which is herein incorporated by reference in its entirety) will be made to evaluate their performance in the fed and/or fasted state.

1. A formulation for oral transmucosal administration of at least one tocotrienol or derivative thereof, comprising:
a first composition comprising
at least one tocotrienol or a derivative thereof,
starch or a derivative thereof, and
silicon dioxide; and
a second composition comprising one or more excipients,
wherein the first composition and the second composition are combined to form the formulation.

2. The formulation of claim 1, wherein the one or more excipient comprises a mucoadhesive polymer selected from the group consisting of lectin, an acrylate, a hyaluronic acid, an alginate, a gellan gum, a poloxamer, a polyethylene glycol, a pectin, a starch, a sulfated polysaccharide, a gelatin, a chitosan, a Carrageenan, and a cellulose derivative and combinations thereof.

3-5. (canceled)

6. The formulation according to claim 1, wherein the formulation is a solid dosage form.

7. (canceled)

8. The formulation according to claim 6, wherein the solid dosage form is a powder or a tablet.

9. The formulation according to claim 1, wherein the at least one tocotrienol is selected from the group consisting of alpha-tocotrienol, beta-tocotrienol, gamma-tocotrienol, delta-tocotrienol and combinations thereof.

10. (canceled)

11. The formulation according to claim 1, wherein the starch or a derivative thereof is tapioca dextrin or modified food starch.

12. (canceled)

13. The formulation according to claim 1, wherein the first composition comprises between 0.1%-2.5% w/w silicon dioxide.

14. The formulation according to claim 1, wherein the first composition comprises between 40%-60% w/w starch or derivative thereof.

15. The formulation according to claim 1, wherein the first composition comprises between 2 to 50% w/w total tocotrienol.

16. (canceled)

17. The formulation according to claim 1, wherein the formulation comprises mannitol.

18. The formulation according to claim 1, wherein the formulation comprises or consists of one or more tocotrienol, silicon dioxide, dextrin, polyethylene glycol 8000, partially pregelatinized corn starch, mannitol, a flavour and a sweetener.

19. (canceled)

20. A process for preparing a formulation for oral transmucosal administration of at least one tocotrienol or derivative thereof, comprising:
(a) combining at least one tocotrienol or a derivative thereof, silicon dioxide and starch or a derivative thereof to form a first composition; and
(b) combining the first composition with one or more excipients.

21. The process of claim 20, wherein the one or more tocotrienol is an oil.

22. The process of claim 20, wherein combining the first composition with one or more excipients comprises mixing or agitation to form a homogeneous and free flowing powder.

23. The process according to claim 20, wherein combining the first composition with one or more excipients comprises mixing at a speed of 25 to 35 rpm for 20 to 30 minutes.

24. The process according to claim 20, further comprising:
(c) forming a solid dosage form selected from a lozenge, a pill, a tablet, a capsule, a membrane, a strip, a patch, a film, or a powder.

25. A method of treating or preventing a disease or condition amenable to treatment with a tocotrienol comprising transmucosal administration of the formulation according to claim 1.

26. The method according to claim 25, wherein the disease or condition is selected from the group consisting of post exercise muscle soreness, delayed onset muscle soreness, muscle recovery after exercise, maintenance of peak muscle power, fibrosis, hypertension, inflammation, stroke, cancer, elevated cholesterol and/or triglycerides; baldness, hypertrophy and a condition resulting from radiation exposure.

27. A method of stabilizing and/or controlling blood glucose levels in a subject comprising transmucosal administration of the formulation according to claim 1.

28. A method of improving exercise endurance, exercise endurance, muscle recovery and/or maintenance of peak muscle power in a subject comprising transmucosal administration of the formulation according to claim 1.

29-36. (canceled)
